(19) United States
(12) Patent Application Publication
(10) Pub. No.: US 2025/0216386 A1
(43) Pub. Date: Jul. 3, 2025

(54) ELECTROCHEMICAL BIOSENSING ELECTRODE BASED ON SIALYLATED GLYCAN FOR THE DETECTION OF NEURAMINIDASES OR SIALYLTRANSFERASES

(71) Applicant: Yissum Research Development Company of the Hebrew University of Jerusalem Ltd., Jerusalem (IL)

(72) Inventors: Shlomo YITZCHAIK, Jerusalem (IL); Mattan HUREVICH, Jerusalem (IL); Israel ALSHANSKI, Shoham (IL)

(73) Assignee: Yissum Research Development Company of the Hebrew University of Jerusalem Ltd., Jerusalem (IL)

(21) Appl. No.: 18/852,769

(22) PCT Filed: Jun. 19, 2023
(86) PCT No.: PCT/IL2023/050634

§ 371 Date: Sep. 30, 2024

(30) Foreign Application Priority Data
Jun. 21, 2022 (IL) 294164

(51) Int. Cl. G01N 33/543; G01N 27/02

(52) U.S. Cl. CPC G01N 33/5438; G01N 27/026

(57) ABSTRACT

The detection of enzymes and other biological materials is accomplished by use of sialylated glycan-based detection units.

### TECHNOLOGICAL FIELD

The invention generally contemplates a biosensor and uses thereof.

### BACKGROUND OF THE INVENTION

Sialic acid (SA) is an important and unique monosaccharide that decorates N-glycans, O-glycans, and gangliosides on the cell membrane (generally referred to as sialosides). SA is important for cell recognition, intercellular communication, and immune system regulation. SA is also a common target for infection recognition. Neuraminidases (NAs) are enzymes that remove SA from sialosides, therefore regulate SA expression. Viral pathogens use NAs or similar proteins as part of the infection process. Viral pathogens can differentiate between cells based on the type of SA and the regiospecificity of its connection to the glycoconjugate.

There are several common approaches for evaluating NAs activity. The first approach is labeled-based. In this case, the substrate can be either fluorescently or metabolically labeled. The labeled substrate undergoes enzymatic reaction or binds the enzyme in a manner that produces a detectable signal such as fluorescence. However, this method requires large quantities of a labeled substrate and hence is limited in the use of hardly accessible sialosides. This is because their synthesis or purification from natural sources is not trivial. The second approach requires an inhibitor that binds the catalytic site and enables structural analyses. In this approach, the binding properties of the enzyme can be studied in a glycan array, for instance, which enables fingerprint patterning of the enzyme. The third approach is use of a sensory interface with a label-free technique. In this case, the substrate is attached to an interface, which produces a detectable signal upon enzyme binding or reaction such as electrochemical signals.

Electrochemical impedance spectroscopy (EIS) is a label-free electrochemical technique for evaluating interactions and biosensing. EIS relies on changes to the interfacial properties, which affect the diffusion through the layer when external RedOx active species is used. EIS is a sensitive technique that requires small amounts of material to produce a detectable signal in the sensory layer. The high sensitivity of EIS can be used for evaluating enzymatic reactions on an interface or protein binding to a substrate in the interface. This produces detectable signals with small amounts of substrate. Additionally, it was shown that enzymes can be detected by impedimetric measurements utilizing affinity to enzyme substrate or allosteric inhibitor and not just by catalytic activity. The ability to study binding and catalysis of enzymes by EIS relies on the chemistry of the interface.

A platform based on bi-antennary N-glycan was developed that enables impedimetric biosensing of sialylation and de-sialylation processes. However, that platform required time-consuming multistep modification on the oxide layer of glassy carbon electrode (GCE).

### General Description

Sialic acid (SA) recognition and hydrolysis are essential features of cellular function and pathogen infectivity. Neuraminidases (NAs) are viral enzymes that detach sialic acid, thereby causing viral infections. Therefore, their inhibition is a prime target for viral infection treatment, whereby the connectivity and type of sialic acid influence the recognition and hydrolysis activity towards the many different neuraminidases. This makes the effort of finding specific viral inhibitors extremely difficult. The common strategies to evaluate neuraminidase activity, recognition and inhibition rely on extensive labeling and require large amounts of sialylated glycans.

The inventors of the technology disclosed herein have developed a novel methodology for distinguishing between different neuraminidases and for evaluating their activity, recognition and inhibition. The platform proposed herein may be utilized in a greater array of analyses for being diverse and highly sensitive. The platform involves synthetic sialylated glycans that differ in the sialic acid origin and connectivity and may thus be used in label-free electrochemical impedance spectroscopy methods to differentiate between different analytes that interact with the sialic acid or the sugar backbone. Such analytes may be neuraminidases. The synthetic sialylated glycans, referred to herein as “sialosides”, can serve as tools for detecting presence and amount as well as evaluating inhibition of neuraminidases binding and enzymatic activity.

The inventors of the technology disclosed herein have synthesized several sialylated glycans, e.g., saccharides such as trisaccharide, having an active functionality, such as an amine, at the terminus on the reducing end and a variability in the sialic acid type and regiochemistry. These sialylated glycans were attached to electrode substrate of different substrate materials, e.g., a substrate of a glassy carbon electrode (GCE) or gold substrate. Differentiation of neuraminidases (NA) could be achieved based on different sialoside types (Neu5Ac vs. Neu5GC) and regiochemistry (2-3 vs. 2-6 linkages). In other words, the platform technology of the invention makes it possible to distinguish between NAs based on the type of sialic acid and its connectivity to the core glycan. As used herein, the regiochemistry broadly refers to the sialosides' structure and not to the NAs. The different NA enzymes, originating from different viral lines and bacterial types, may bind to the sialosides with specific regioselectivity.

The modifications were characterized by EIS, contact potential difference (CPD), contact angle (CA), variable angle ellipsometry (VASE), and X-ray photoelectron spectroscopy (XPS). The modified GCEs and AuEs were exposed to two types of NAs to determine preferential response. Surface characterizations were used to elucidate if a signal received arises from enzymatic activity or binding after exposure to the enzyme. The impedimetric response was further elucidated by applying different concentrations of the NA enzyme in each system. Additionally, the effect of a NA inhibitor on the generated impedimetric signal for exposure to NA was examined.

In most general terms, the invention provides an electrode material having a sialoside functionality, namely an electrode material that is a sialylated glycan (or a glycosylated sialic acid group). The term “electrode material” broadly encompasses a material used to form an electrode for use in detection systems and devices. The electrode material may be provided as a standalone material or associated with an electrode substrate material as known in the art. The electrode substrate may be of any shape and form and is typically conductive. Non-limiting examples of electrode substrate materials include gold substrates and GCE substrates.

The electrode material is a sialoside having a glycan moiety which may be a mono-, di-, tri-, oligo- or a polysaccharide that is associated to a functionality derived from sialic acid (or which is sialic acid). Each of the glycans is given the meaning acceptable in the art. As used herein, the term “monosaccharide” refers to a simple form of a sugar that consists of a single saccharide unit which cannot be further decomposed to smaller saccharide building blocks or moieties, while the “disaccharide” is the smallest repeating backbone unit consisting of two sugar residues. The “trisaccharide” encompasses any sugar formed when three monosaccharides are joined by glycosidic linkages. The terms “oligosaccharides” and “polysaccharides” refer to saccharide polymers containing three to nine sugar residues or 10 or more sugar residues, respectively.

In some embodiments, the glycan is a sugar comprising or consisting a saccharide selected from galactose (Gal), glucose (Glc), and fructose. In some embodiments, the glycan comprises Gal and/or Glc.

In some cases, the glycan is a disaccharide or a trisaccharide, or a higher saccharide such as an oligo- or a polysaccharide comprising one or more Gal and/or Glc units. In some embodiments, the sialoside comprises a glycan having as a core structure the sequence β-D-Gal-(1-4)-β-D-GlcNAc-(1-4))-1-X, wherein X is as defined herein.

The electrode material is tailored for association on a surface region of an electrode capable of biosensing. The electrode material is thus structured to provide selective sensing of an interaction between an agent and the electrode material, wherein the interaction may be detectable by electrochemical impedance spectroscopy (EIS).

Thus, in a first of its aspects, the invention provides an electrode material comprising or consisting or being a sialoside (or a sialylated glycan), wherein the sialoside comprises (or is structured of) a glycan selected amongst monosaccharides, disaccharides, oligosaccharides, oligosaccharides and polysaccharides, as defined herein, and a sialic acid moiety.

In some embodiments, the glycan is a disaccharide, optionally a disaccharide comprising or consisting a core sequence β-D-Gal-(1-4)-β-D-GlcNAc-(1-4))-1-X, wherein Gal is galactose, Glc is glucose and X may be a C₂-C₁₀alkyl, C₂-C₁₀alkenyl, C₂-C₁₀alkynyl, a C₆-C₁₀arylene, or a C₂-C₁₀alkyl-C₆-C₁₀aryl, each being substituted by an atom or a group of atoms, being one or more of halogens (I, Br, Cl or F), hydroxy (OH), thiol (SH), disulfide (—S—S—), cyano (CN), nitro (NO₂), azide (N₃), amine (NH₂, or a primary, secondary or tertiary amine), carboxy (COO), phosphate groups and others.

In some embodiments, in the core sequence, X is an alkyl substituted by an amine group, wherein the alkyl has between 2 and 10 carbon atoms, i.e., C₂-C₁₀alkyl-NH₂.

In some embodiments, the glycan is a disaccharide comprising or consisting a core sequence β-D-Gal-(1-4)-β-D-GlcNAc-(1-4))-1-X-amine, wherein X is C₂-C₁₀alkyl.

In some embodiments, the glycan is a disaccharide comprising or consisting a core sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others.

In some embodiments, the glycan is a disaccharide comprising or consisting a core sequence being β-D-Gal-(1-4)-β-D-GlcNAc-(1-4))-1-pentylamine or β-D-Gal-(1-4)-β-D-GlcNAc-(1-4))-1-hexylamine.

In some embodiments, X may be a C₂-C₁₀alkyl, C₂-C₁₀alkenyl, C₂-C₁₀alkynyl, a C₆-C₁₀arylene, or a C₂-C₁₀alkyl-C₆-C₁₀aryl, each being substituted by an atom or a group of atoms capable of associating to a surface region of the substrate, e.g., an electrode substrate. In some embodiments, the surface associating atom or group of atoms may be hydroxy (OH), thiol (SH), disulfide (—S—S—), azide (N₃), amine (NH₂, or a primary, secondary or tertiary amine), carboxy (COO), phosphate groups and others.

In some embodiments, sialosides of the invention may be regarded sialylated trisaccharides, for containing three saccharide units, one of which derived from sialic acid.

Also provided is an electrode for use in electrochemical impedance spectroscopy (EIS), the electrode having a conductive surface associated to a sialoside (or a sialylated glycan) as disclosed herein.

In some embodiments, the electrode is a gold or a GCE electrode or an electrode useful in electrochemical impedance spectroscopy (EIS), whereby a surface region of the electrode is associated with a sialoside. In some embodiments, the sialoside has a glycan moiety which may be a mono-, di-, tri-, oligo- or a polysaccharide that is associated to a functionality derived from sialic acid (or which is sialic acid).

Thus, the invention further provides an electrode comprising a gold or a GCE substrate, said substrate being associated with a sialoside, as defined herein.

In some embodiments, the sialoside comprises a glycan comprising or consisting a core sequence β-D-Gal-(1-4)-β-D-GlcNAc-(1-4))-1-X-amine, wherein X is as defined herein. In some embodiments, X is a C₂-C₁₀alkyl, substituted as disclosed herein, e.g., by an amine or a surface binding functionality.

In some embodiments, the sialoside comprises a glycan selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others.

In some embodiments, the sialoside comprises the core sequence β-D-Gal-(1-4)-β-D-GlcNAc-(1-4))-1-pentylamine or β-D-Gal-(1-4)-β-D-GlcNAc-(1-4))-1-hexylamine.

As may be understood, the sialoside provided on the electrode surface acts as a barrier to charge transport from the conductive electrode substrate to the solution.

In some embodiments, the electrode is one suitable for biosensing.

In some embodiments, the electrode material of the invention, as used, for example, in electrodes of the invention, is a sialoside, namely a sialylated glycan, such as a sialylated trisaccharide, wherein the group derived from sialic acid that is associated to the glycan, e.g., a disaccharide, via a linker moiety having an amine at the terminus on a reducing end and variability in the sialic acid moiety. The sialoside may comprise any human sialic acids or any monkey type sialic acids. The sialic acids may be selected from N-acetylneuraminic acid (Neu5Ac) and N-glycolylneuraminic acid (Neu5Gc).

In some embodiments, the sialic acid has an acetyl at position 5, e.g., Neu5Ac, with connectivity 2,6 (H6) and 2,3 (H3) (being human sialic acids). In some embodiments, the sialic acid has a hydroxy acetyl at position 5, e.g., Neu5Gc, with connectivity 2,6 (M6) and 2,3 (M3) (being monkey sialic acids). Other types of sialic acids may also be used.

Thus, in some embodiments, the electrode material is one or more sialosides having the Neu5Ac and/or Neu5Gc groups. In some embodiments, the electrode material is or comprises a sialoside herein designated H6, H3, M6, and M3, respectively having the structures:

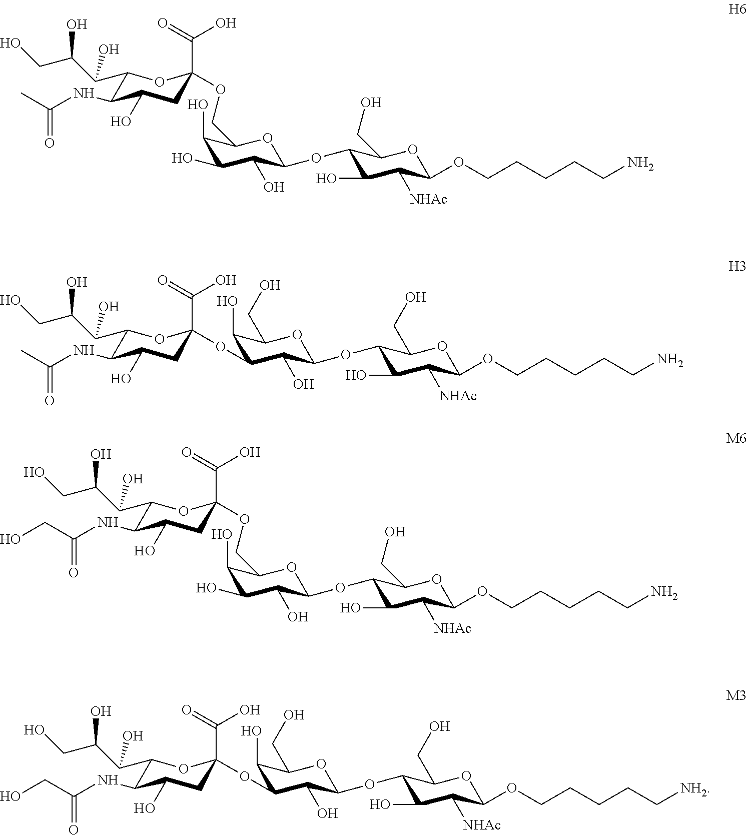

In some embodiments, the electrode of the invention is an electrode having a biosensitive surface region associated with an electrode material selected amongst sialosides herein designated H6, H3, M6 and M3. The biosensitive surface region may be any surface region of the electrode, or the complete surface of the electrode. In some cases, the electrode is suitable for EIS.

A biosensitive surface may be achievable by any chemical association protocols known in the art. Where the electrode, for example, is a gold electrode, association of the electrode material to the gold surface may be achievable by lipoic acid chemisorption and subsequent amidation with the sialylated glycan, e.g., H3, H6, M3, or M6. In another example, where the electrode is a glassy carbon electrode (GCE), electrochemical grafting of the sialylated glycan may be used.

Without wishing to be bound by theory, in order to achieve a robust association of the sialoside molecules onto a surface region of the electrode, the association typically involves chemical adsorption (or chemisorption) via surface binding groups which may be part of the sialoside structure or may be associated therewith before or during the deposition process. The surface binding groups may be amines, carboxylic acids, thiols, disulfides or generally S-containing functionalities, phosphates and generally P-containing functionalities, and others, as disclosed herein and as known in the art. Surface association may form a monolayer of the sialosides on a surface region or the complete surface of the substrate. The monolayer may be homogenous in e.g., composition and height, or may be heterogeneous, namely comprising sialosides of different compositions.

In some cases, the monolayer may be formed of a mixture of different sialoside populations or types, wherein each population or type differs in composition from the other. The difference may be in the sialic acid derived moiety, the glycan, the terminal chain, and/or the surface binding group. For example, where the sialoside is a trisaccharide having the core structure β-D-Gal-(1-4)-β-D-GlcNAc-(1-4))-1-X, wherein X is as defined herein, the monolayer may comprise sialosides having the aforementioned core structure, wherein X is an alkylamine of various lengths. In other words, a monolayer may be comprised of sialosides of different compositions/lengths, wherein for each sialoside type the X is differently selected from C₂-C₁₀alkylamine.

In some embodiments, the electrode is surface modified with a monolayer of at least one sialoside population.

In some embodiments, the electrode is a gold electrode or a GCE electrode associated with a monolayer of at least one sialoside population.

Thus, further provided is a modified electrode, wherein the electrode surface is modified by a sialylated glycan, wherein the glycan is selected from monosaccharides, disaccharides and trisaccharides, associated to a sialic acid moiety being Neu5Ac or Neu5Gc. In some embodiments, the sialylated glycan is one or more of herein designated H3, H6, M3, or M6.

In some embodiments, the electrode is an array of electrodes comprising electrodes of the same type and/or electrodes modified in the same way, e.g., modified with the same sialylated glycan material. In some embodiments, an array of electrodes is provided which comprises two or more different types of electrodes.

In some embodiments, the array comprises gold-based electrodes. In some embodiments, the array comprises a GCE-based electrode. In some embodiments, the array comprises a gold-based electrode and a GCE-based electrode.

The invention further provides a detection device, e.g., an electrochemical impedance detection device, comprising an electrode according to the invention. The device may be in a form of a biosensor device which employs an electrode having a sialylated glycan surface in combination with impedance measuring elements integrated into the device. The sialylated glycan of the disclosure may be incorporated onto the surface of the electrode and a biological sample may then be flown or brought into contact with the surface of the electrode. A change in the detected impedance generally indicates agent binding or agent interaction to said sialylated glycan surface.

The detection device of the invention, being in some configurations a device for measuring a change in an electrochemical impedance of a surface, is structured with an electrode of the invention to enable analysis of interfacial properties related to biorecognition events that occur on the surface of the electrode in view of an interaction of the sialylated electrode surface with an agent or analyte, e.g., which presence and/or concentration is to be determined. The interaction which may be biocatalytic (resulting in dissociation of a sugar unit from the sialylated electrode surface) or biorecognition (resulting in an association between the sialylated surface and the agent) may be used to derive information as to the occurrence of the interaction, the type of interaction, the agent interacting with the surface, the degree and rate of interaction, the concentration of the agent present, presence of different agents, and others. Thus, the device of the invention may be exploited in a variety of fields, for achieving an effective quantitative and qualitative detection of agents such as pathogens, DNA, cancer-associated biomarkers, sugar-interacting agents, enzymes, antibodies, aptamers, cells and others.

In some embodiments, devices of the invention may be used for the detection of enzymes such as NAs in a sample. In some embodiments, the NA is a viral NA.

A difference in an electrical signal as compared to a base signal measured prior to a potential interaction between an agent and the modified electrode surface may occur due to kinetic binding of the agent to the sialylated surface or due to a change in the composition of the sialylated surface due to dissociation of the sugar backbone. As a result, electron transfer/charge transfer resistance is produced, representing the amount or concentration of bound agents or the degree of dissociation. Thus, direct and rapid determination of both biomolecular recognition actions as well as biocatalytic events is possible.

Devices of the invention may be provided as bench-top detection devices or as lab-on-a-chip devices.

Where a device of the invention comprises two or more sialylated electrodes or an array of such electrodes, both high sensitivity and high selectivity may be achievable as both biocatalytic and biorecognition events may be detected and reported. Various types of NAs can be differentiated by preferential activity with sialylated substrates, as exemplified herein. As further demonstrated, a device of the invention may be used to electrochemically evaluate enzyme preference to the sialylated surface and distinguish between different NAs. As NAs are enzymes that detach sialic acid, their inhibition is a prime target for viral infection treatment, whereby the connectivity and type of sialic acid influence the recognition and hydrolysis activity towards the NAs. Thus, use of a sialylated surface according to embodiments of the invention may be used to screen for inhibitors of NAs.

In fact, methods utilizing sialylated electrodes and devices of the invention may be used for determining presence of agents not only in the biomedical arena. In most general terms, any interaction between a sugar, represented by the sialylated glycan and an analyte may be detected.

In another aspect, the invention thus provides a method for determining an interaction between a sugar, namely the sialylated glycan, e.g., sialylated trisaccharide, and an agent/analyte, wherein the method comprises contacting a sialylated electrode surface, as disclosed herein, with a sample containing or suspected of containing said agent and determining a change in an impedance signal generated from a base signal obtained for a control sample. The change is likely to result from an interaction, as disclosed herein, between the sugar, i.e., the sialylated surface of the electrode and the agent.

The “agent” which detection is desired may be any such material which can chemically or physically interact with the sialylated glycan associated to a surface region of the electrode to produce a measurable resistance or a measurable change in a surface impedance. As noted herein, the agent may interact with the sialylated glycan by binding to a region of the sugar moiety or by directly or indirectly causing a structural change in the sialylated glycan, e.g., by bond scission. Depending on the sample or environment in which the agent is present, or suspected of being present, the agent may be any solid, liquid or gaseous material of an environmental origin, a biological origin, a natural origin, a synthetic origin or of an unknown origin.

Where elements of the invention (such as electrodes and devices) are used in the medical arena, biochemical or chemical agents may be detected. Such include inhibitors, chemical ablation agents, toxins, pathogens, immunomodulators, cytokines, cytotoxic agents, chemotherapeutic agents, drugs and therapeutic agents, DNA, cancer-associated biomarkers, peptides and proteins, enzymes, antibodies, aptamers, cells, glycans and glycol-conjugates and others. Where elements of the invention are used in an environmental setting, e.g., for detection of environmentally present agents, chemical such as toxins, toxic gases, free radicals, body odors and body odor volatiles, volatile organic compounds, metal ions and metalloproteins and others may be detected.

The agent may be presented to a surface of an electrode of the invention in a carrier medium, which does not by itself generate a measurable single, or which can be used as a control or background signal for determining presence of an agent carried in a sample of similar constitution. The medium may be water or containing water. Samples of unknown composition may be diluted in a buffer and tested.

As generally disclosed herein, methods of the invention comprise contacting an electrode, as defined herein, with a sample containing or suspected of containing an agent and determining a change in an impedance signal generated from a base signal obtained for a control sample. The control sample used for determining a set point, a threshold or a background signal from which a signal may be attributed to the agent may be determined, may be a carrier medium identical to that of the sample known not to contain the agent, an identical sample known not to contain the agent, or generally any agent-free sample. For example, where presence of a viral NA is a biological sample, e.g., blood sample, is desired, a blood sample known not to contain the viral NA may be used as a control.

Similarly, for determining concentration of an agent or an improvement in a medical treatment following or during treatment, e.g., antiviral treatment, a subject's blood samples may be testes on numerous occasions during a period the subject is undergoing the medical treatment to detect changes in the concentration of the NA, as determined by a change in impedance, whereby such a change may indicate a reduction in the amount of NA present in the blood of the subject. A determination of concentration may be based on predetermined studies using varying known concentrations of the agent to be detected, as known and practiced in the art.

The invention further provides a method for determining an amount or a concentration of an agent in a sample, the agent being capable of interacting with a sialylated glycan, e.g., sialylated trisaccharide, surface (an electrode surface coated or associated with the glycan), the method comprising contacting said surface with the sample, measuring a change in an impedance signal relative to an impedance signal measured for one or more control samples having known concentrations of the agent and determining concentration of the agent in the sample.

Also provided is a method of screening for a viral inhibitor, the method comprising contacting a sialylated glycan, such as a sialylated trisaccharide electrode surface, as disclosed herein, with a potential viral inhibitor in presence of a viral pathogen or a neuraminidase (NA) and determining a change in an impedance signal generated upon contact with the glycan provided on the electrode surface, wherein a change in the signal from a base signal obtained for a control sample not containing the pathogen or NA and/or not containing the potential viral inhibitor indicates inhibition or absence of inhibition of the pathogen or NA by the inhibitor. In other words, where in the presence of the potential viral inhibitor a change in the signal is not observed, the indication may be successful partial or full inhibition of the pathogen or NA. Where a change in the signal is observed, that change may support that the potential inhibitor is in fact not capable of inhibition.

The change in the impedance signal as compared to a base signal generated for a control sample provides an indication of an interaction between an agent and the sialylated glycan surface. The change on the signal may be an increase or a decrease in the impedance signal relative to a base line of the glycated surface under buffered solution (enzyme free). An increase of the impedance signal may suggest adsorption or association of the agent, e.g., an enzyme, to the sialylated glycan (via biorecognition pathways), while a decrease in the signal may suggest dissociative interaction (via biocatalytic pathways).

In some embodiments, methods and systems of the invention are tailored for biomedical uses, including screening for active drugs, determining presence or absence of biological markers in a sample, presence or absence of pathogens or toxins in a sample, etc.

In some embodiments, methods and systems of the invention may be used for screening for enzyme inhibitors. An enzyme inhibitor may be any material which reduces a rate of an enzyme catalyzed reactions by interfering with the enzyme in some way. In some embodiments, the enzyme inhibitor is an antiviral, antibiotic, anticancer drug, e.g., which screening is desired.

In some embodiments, the enzyme inhibitor is an inhibitor of NA, as defined herein.

In some embodiments, the enzyme inhibitor is an inhibitor of sialyltransferase.

The invention further provides a kit comprising a sialoside of the invention and instructions permitting association thereof onto a surface region of a substrate.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D provide structures of trisaccharides used in accordance with some embodiments of the invention: A) 2,6-Neu5Ac-Gal-GlcNAc trisaccharide (H6), B) 2,3-Neu5Ac-Gal-GlcNAc trisaccharide (H3), C) 2,6-Neu5Gc-Gal-GlcNAc trisaccharide (M6), D) 2,3-Neu5Gc-Gal-GlcNAc trisaccharide (M3).

FIGS. 2A-B provide A) Synthesis of donor 8. Reagents and conditions: (a) i) Ac₂O, pyridine, rt, 12 h; ii) Cs₂CO₃, AllylBr, DMF, 40° C., 4 h, 69% over two steps; (b) p-Thiocresol, BF₃·OEt₂, CH₂Cl₂, rt, 24 h, 78%; (c) Boc₂O, DMAP, THF, 60° C., 4 h, 85%; (d) NaOMe, AllylOH, rt, 4 h, 56%; (e) i) TFA/CH₂Cl₂ (1:1, v/v), rt, 3 h ii) NO₂C₆H₄OCOCl, NaHCO₃, H₂O/MeCN (2:1, v/v), 0° C., 4 h, 47% over two steps; (f) Ac₂O, pyridine, rt, 12 h, 84%; (g) Acetoxyacetyl chloride, DIPEA, CH₂Cl₂, 0° C. to rt, 2 h, 83%; (h) NIS, TfOH, dibutyl phosphate, CH₂Cl₂, 0° C., 6 h, 74%. B) Synthesis of M6 and M3 trisaccharides. Reagents and conditions: (i) 12, TMSOTf, CH₂Cl₂, −50° C., 2 h, 72%; (j) 19, NIS, TfOH, CH₂Cl₂, −20° C., 2 h, 21: 71%; 26: 68%; (k) Zn, THF/AcOH/Ac₂O (3:2:1, v/v), rt, 4 h, 22: 65%, 27: 70%; (1) 1,2-ethanedithiol, DBU, CH₂Cl₂, 0° C., 2 h, 23: 74%; 28: 75%; (m) i) LiOH, THF/H₂O/MeOH (2:2:1, v/v), rt, 12 h; ii) Pd(OH)₂/C, H₂, H₂O/MeOH (3:1, v/v), rt, 48 h, 24: 63%; 29: 59%, over two steps. (n) i) 17, TMSOTf, CH₂Cl₂, −50° C., 2 h; ii) Ac₂O, pyridine, rt, 12 h, 49% over two steps.

FIGS. 3A-B depict A) electrografting process of GCE with i) amino terminated trisaccharide applying 5 cycles of scans from 0.6 to 1.2 V at scan rate of 0.01 V/s B) modification of AuE with trisaccharides in two steps: ii) self-assembly of LPA and iii) coupling the amino terminated trisaccharide with the LPA using COMU.

FIGS. 4A-D depict A) Nyquist plot of Impedimetric response of H3 before and after exposure to 3NACP. B) Normalized R_{CT} for the response of GCE H6 and H3 after exposure to 3 mU/mL 6NAAU or 3NACP. C) Nyquist plot of Impedimetric response of H3 before and after exposure to 3NACP. D) Normalized R_{CT} for the response of GCE H6 and H3 after exposure to 3 mU/mL 6NAAU or 3NACP. Errors are the standard deviation of 5 electrodes.

FIG. 5 provides a heat map of the response magnitude after exposure the substates to the enzyme in different platforms. The response is in Normalized R_{CT}.

FIGS. 6A-B provide A) Normalized R_{CT} of concentration dependent response to the 3NACP with GCE-H3. B) Normalized R_{CT} of concentration dependent response to the 3NACP with AuE-H3.

FIGS. 7A-B provide A) Normalized R_{CT} of impedimetric response of GCE-H3 to 0.3 mU/mL 3NACP with different concentrations of Oseltamivir. B) 1/Normalized R_{CT} of impedimetric response of AuE-H3 to 3 mU/mL 3NACP with 1 μM Oseltamivir and without.

FIG. 8 provides a schematic summary of the sensing ability using AuE and GCE. AuE modified with the trisaccharide is responsive to the enzymatic reaction while GCE modified with the trisaccharide is responsive to enzyme binding.

FIGS. 9A-B depict A) Influenza utilizes Hemagglutinin to target sialylated glycan on cell surface and uses neuraminidase to detach from the cell by cleaving sialic acid. B) Systems used in this work to target Influenza neuraminidase.

FIG. 10 depicts modified GCE and AuE with the trisaccharides according to some embodiments of the invention.

FIGS. 11A-B shows Nyquist plot of response to H1N1 NA by A) GCE-H3 and by B) AuE-H3 where green plot is prior to exposure and red is after exposure.

FIGS. 12A-B depicts normalized R_{CT} response to H1N1 and H3N2 NA by A) GCE modified with sialosieds B) AuE modified with sialosieds. The standard deviation is based on response of 5 electrodes.

FIG. 13 shows a heatmap of Log(Normalized R_{CT}) for the response of different NA with the presented sensory layers.

FIGS. 14A-E provide a Radar plot of |Log (Normalized R_{CT})| for the response of H1N1 (A), H3N2 (B), H5N1 (C), NACP (D), and NAAU (E) with the presented sensory layers.

FIG. 15 provides normalized R_{CT} response of AuE-H3 to H3N2 NA in presence and absence of Viral NA inhibitors.

### DETAILED DESCRIPTION OF EMBODIMENTS

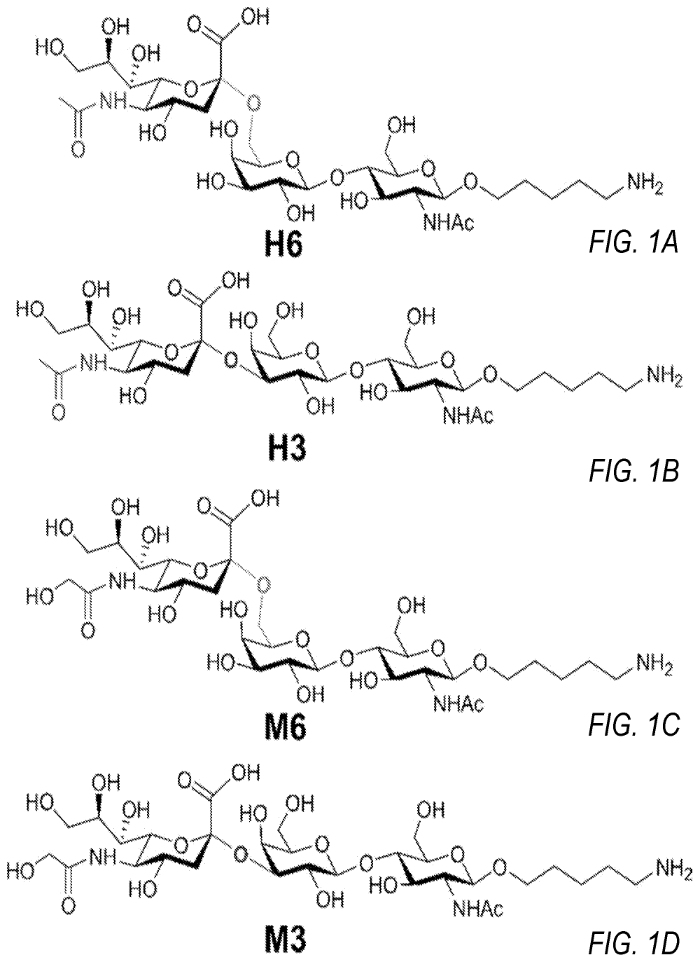

Various types of neuraminidases (NA) can be differentiated by preferential activity with sialylated substrate. Four trisaccharide substrates were synthesized via multistep processes to allow high control of the regiochemistry on a common core sialosides (FIG. 1). The four substrates containing the same core structure of 6-(β-D-Gal-(1-4)-β-D-GlcNAc-(1-4))-1-amine with two types of sialic acid and two types of connectivity. Two human sialosides are known to contain acetyl at position 5, namely Neu5Ac, with connectivity 2,6 (H6) and 2,3 (H3) and there are two monkey type sialosides with hydroxy acetyl at position 5, namely Neu5Gc with connectivity 2,6 (M6) and 2,3 (M3) (FIG. 1). The chemical synthesis of sialic acid glycans is a formidable synthetic challenge due to its instability, difficulties in α-glycosylation and low reactivity. Using a similar synthetic strategy sialic acid donor, Neu5Ac analogs (H6 and H3) were synthesized (FIG. 1). However, the synthesis of Neu5Gc glycans using these methods is still a challenging task. Therefore, an enzymatic method has been extensively used in the synthesis of complex sialylated-glycans.

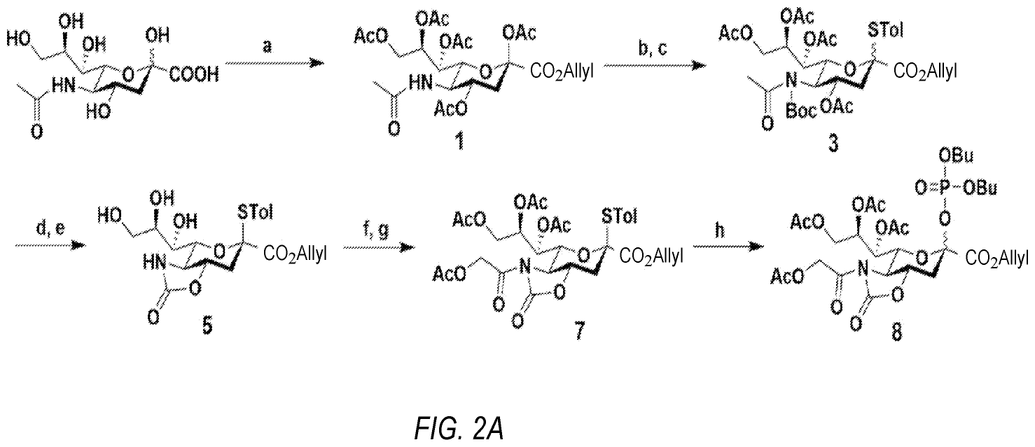

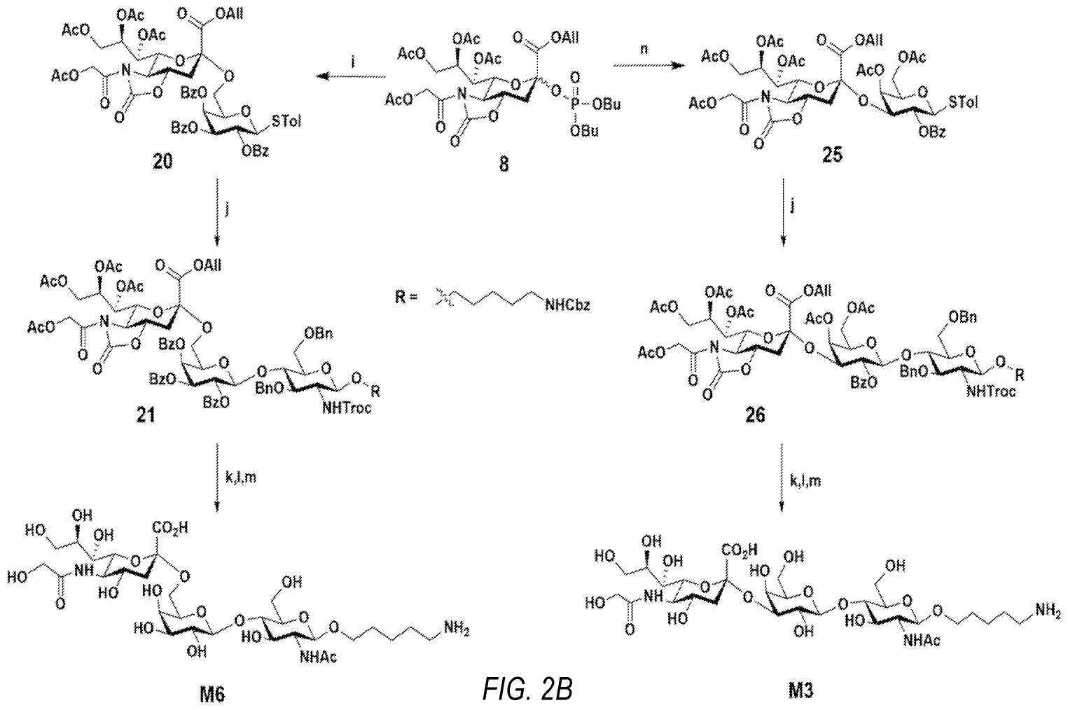

In the synthetic strategy disclosed herein, two key steps were adopted for synthesizing Neu5Gc glycans: (a) sialic acid glycans were developed using allyl ester instead of traditional methyl ester-ligand to avoid harsh deprotection conditions, which may cleave α-sialyl linkage; (b) a labile method was developed to deprotect oxazolidinone ring to control the selective N-glycolyl substitution. Neu5Gc glycans were obtained from orthogonally protected sialic acid donor 8 and sequentially glycosylated to galactose and glucose building blocks under standard glycosylation conditions FIG. 2. The sialic acid donors were obtained by single step peracetylation and allyl-esterification of sialic acid, followed by p-thiocresol glycosylation and Boc-protection. Deacetylation of 3 in the presence of sodium methoxide and allyl alcohol, followed by oxazolidinone formation and peracetylation yielded desire donor 5, which was treated with acetoxyacetyl chloride to obtain Neu5Gc 7 thio-donor. Finally, glycosylation of 7 with dibutyl phosphate in the presence of N-iodosuccinimide (NIS) and trifluoromethanesulfonic acid (TfOH) yielded the desire sialic acid donor 8 in an excellent yield (FIG. 2).

To achieve α(2-6) and α(2-3) glycosylated sialic acid disaccharides 20 and 25, two different galactose building blocks 12 and 17 were synthesized in 5 and 8 steps from D-galactose (ESI). The glucose building block 18 was synthesized and glycosylated with Cbz-amine protected linker using standard glycosylation conditions to obtain 82% of 19 (ESI). The sialic acid disaccharides (20, 25) was obtained by glycosylating the sialic acid donor 8 with 12 and 17 acceptors in the presence of TMSOTf at −50° C. in DCM solvent (FIG. 2B). In the case of α-(2-3) disaccharides, the glycosylated product was again reacted with acetic anhydride to block 4-OH group on galactose residue. Then glycosylation of disaccharide thio-donors (20 and 25) with 19 acceptor was carried out with NIS/TfOH at −20° C. gave protected trisaccharide in moderate to good yield (FIG. 2B). To accomplish the final deprotected M6 and M3, the correct order of deprotection is critical to obtain Neu5Gc analogs. It was found that the oxazolidinone deprotection prior Troc-removal resulted in partial deprotection of Troc. In addition, the global deprotection of oxazolidinone, acetate and benzoyl group using strong basic conditions also resulted complete deprotection of glucose N-acetate. Thus, Troc-protection removal and acetylation are the first necessary step to maintain the N-glycolyl group. This was followed by selective oxazolidinone deprotection using 1,2-ethanethiol and DBU mixture, followed by global deprotection using lithium hydroxide, followed by hydrogenolysis yielded (M6) and (M3). The trisaccharides (H6, H3, M6, M3) were synthesized with a primary amine at the terminus of the extending linker (FIG. 1). This enables electrochemical grafting on glassy carbon electrode (GCE) or amidation with lipoic acid on modified Au electrode (AuE).

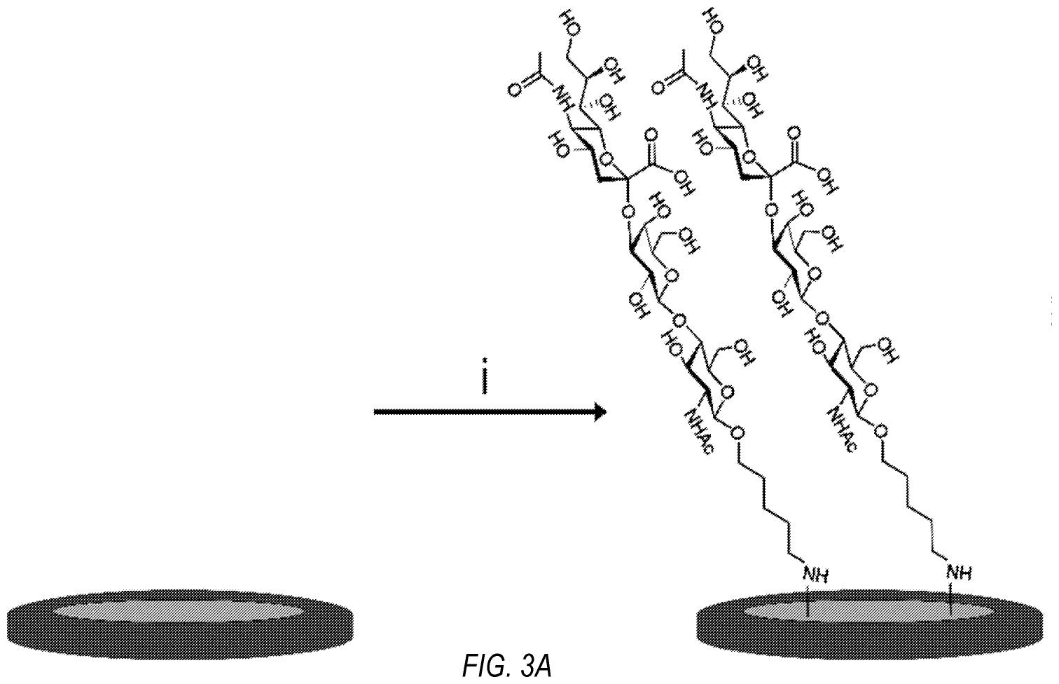

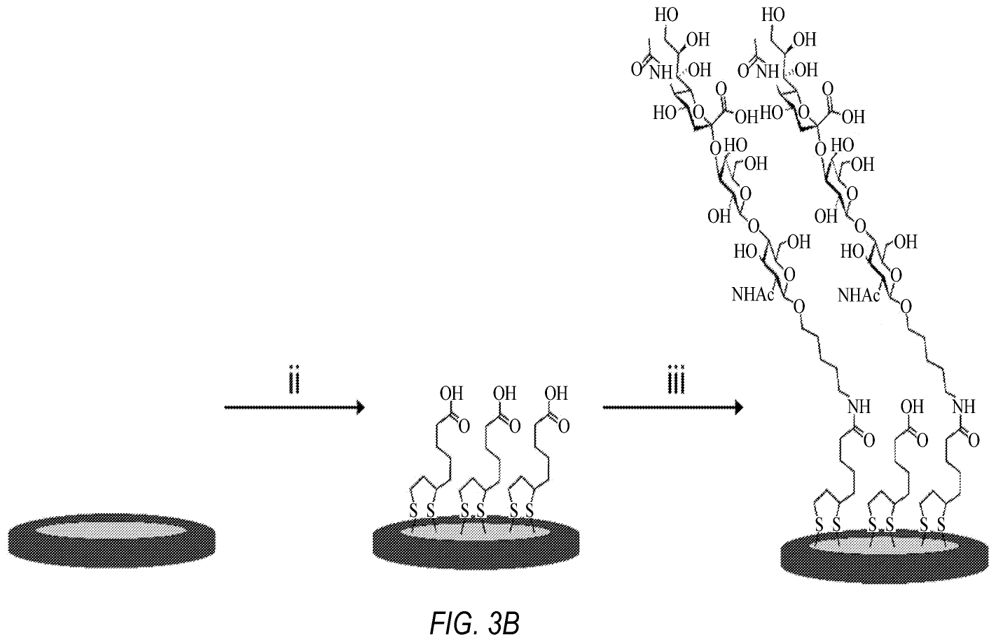

The substrates H6, H3, M6, and M3 were electrochemically grafted with the GCE by applying 5 cycles of CV with a scan rate of 0.01 V/s from 0.6 to 1.2 V referenced to Ag/AgCl (3M KCl) electrode to give GCE-H6, GCE-H3, GCE-M6, and GCE-M3 respectively (FIG. 3A). This resulted in charge transfer resistance (R_{CT}) increase to an approximately value of 5000 after the deposition suggests grafting with the glycan. To support this claim, deposition with the same condition was performed on glassy carbon plates (GCP) with H6 to give GCP-H6. These plates were characterized by CPD and CA analyses. A decrease in CA from 750 to 550 suggests the addition of hydrophilic molecules on the surface. The increase in V_{CPD} from −315 to −106 mV (ΔCPD=+209 mV) suggests the addition of negative charges, which are correlated with the deprotonated carboxylates of the sialic acid. The collective data suggest that the saccharides were electrografted on the GCE. Therefore, they can be further evaluated for impedimetric analyses.

Another system was prepared by modifying AuE to give broader insight on the enzymatic reaction in an interface. AuE were modified with lipoic acid chemisorption and amidation with H3, H6, M3, and M6. Each step of modification showed an increase in the R_{CT} suggesting modification of the AuE. VASE analyses of the modification and Au surfaces showed the formation of a layer with a thickness of 5 Å, CA of 60°, and V_{CPD} of −192 mV following chemisorption of LPA. Coupling of the trisaccharide resulted in an increase of the layer thickness to 13 Å and decrease of V_{CPD} to −232 mV (ΔCPD=+40 mV) suggesting the formation of a sensory layer with the saccharide on AuE. However, the hydrophobicity of the layer remained unchanged with CA of 60°.

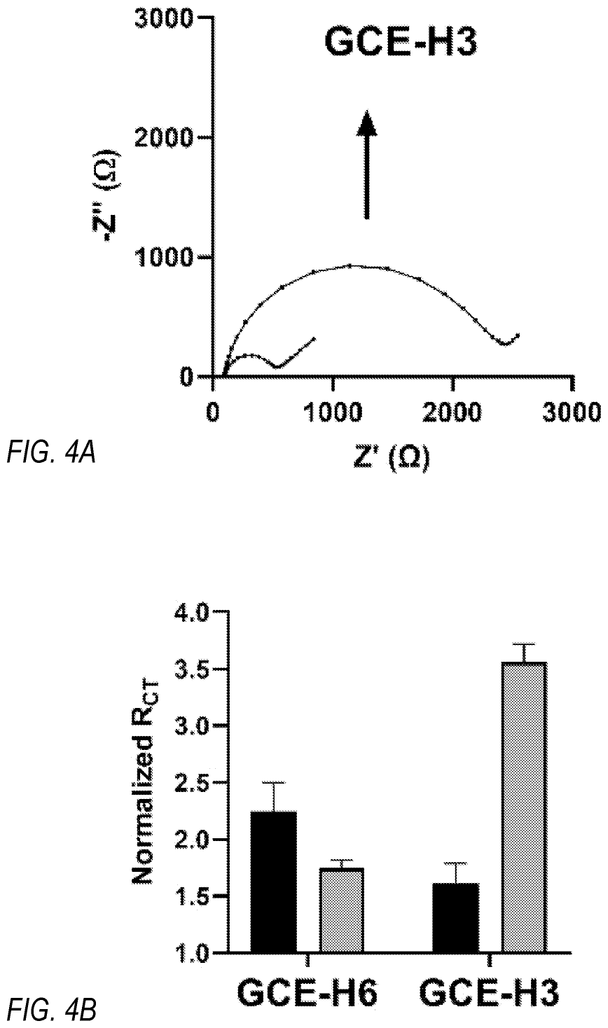

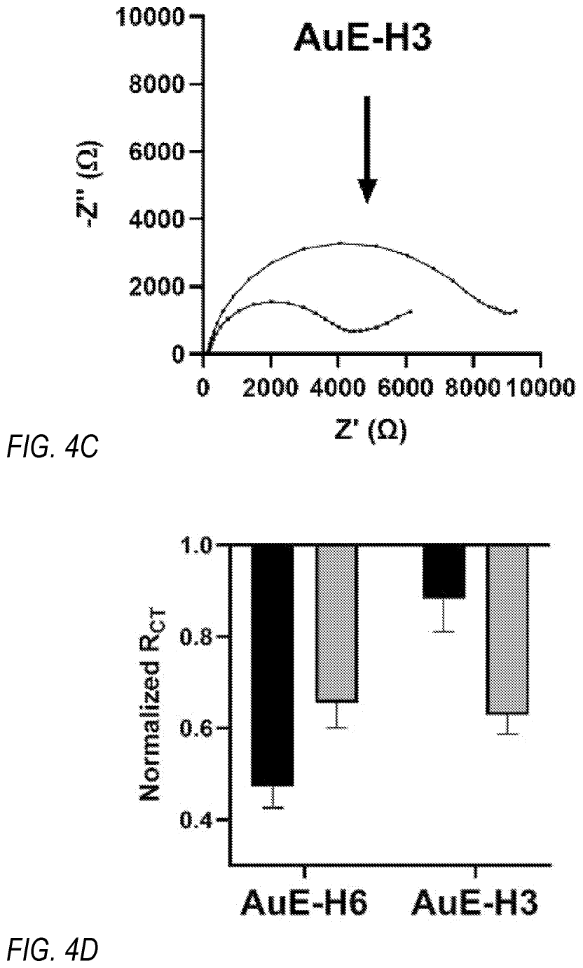

EIS analyses were performed on GCE-H6 and GCE-H3 prior and after exposure to 3 mU/mL of two types of NA and the R_{CT} was normalized to the initial value obtained from the Nyquist plot (FIGS. 4A and B). The first NA, which has preference for 2,3 bond cleavage, is isolated from Clostridium perfringens (3NACP) and the second NA, which prefers 2,6 bond cleavage, is isolated from Arthrobacter ureafaciens (6NAAU). GCE-H6 showed a preferential response to 6NAAU while GCE-H3 showed a preferential response to 3NACP. These results are in line with the reported enzyme sialosides specificity.

EIS analyses were performed on AuE-H6 and AuE-H3 prior to and after exposure to 3 mU/mL of the two types of NA (FIG. 4C). In the case of the modified AuE, a decrease in the R_{CT} value was observed with the same substrates attached to GCE. The response was summarized in FIG. 4D. The relative responses of AuE-H6 and AuE-H3 were preferential to 6NAAU and 3NACP respectively. This shows that there is a preference of electrode bound substrate to the proper enzyme independently of the surface used and the signal behavior and that EIS may be used to evaluate electrochemically enzyme preference and distinguish between NA.

Surface characterization techniques were used to evaluate the source of the impedimetric signal resulting from exposure of enzyme to substrate on GC and Au. XPS, CPD, and CA analyses were performed on GCP-H3 before and after exposure to 3NACP. A relative increase in N1s peak at 400.4 eV related to amides and C1s at 288.9 eV related to carbonyls suggests that the enzyme is adsorbed on the surface of the modified GCP. Change in CPD by −198 mV to a value of −304 mV after exposure to 3NACP, which is very close to the initial value of −315 mV, implies that there is an addition of a dipole that cancels the previous effect. This non-oriented dipole can be related to the addition of the enzyme. An increase of CA to 82° also suggests a change in the interfacial properties. Assuming that there is an addition of the enzyme on the surface, the increase in hydrophobicity can be related to the adsorption of the protein with hydrophobic functional groups pointing towards the interface. VASE analyses of exposure of Au-H3 to 3NACP showed no change to the layer thickness, which remains 13 Å thick, and a small increase in hydrophobicity to 660 and V_{CPD} to −215 mV. Additionally, XPS showed no addition of amide bonds related to the enzyme backbone after exposure. These analyses indicate that the enzyme is not adsorbed to the gold layer, hence, the observed response can be related to enzymatic reaction. The surface characterization analyses imply that the observed impedimetric response on GCE is related to specific adsorption of the enzyme whereas the response on AuE is related to the enzymatic reaction.

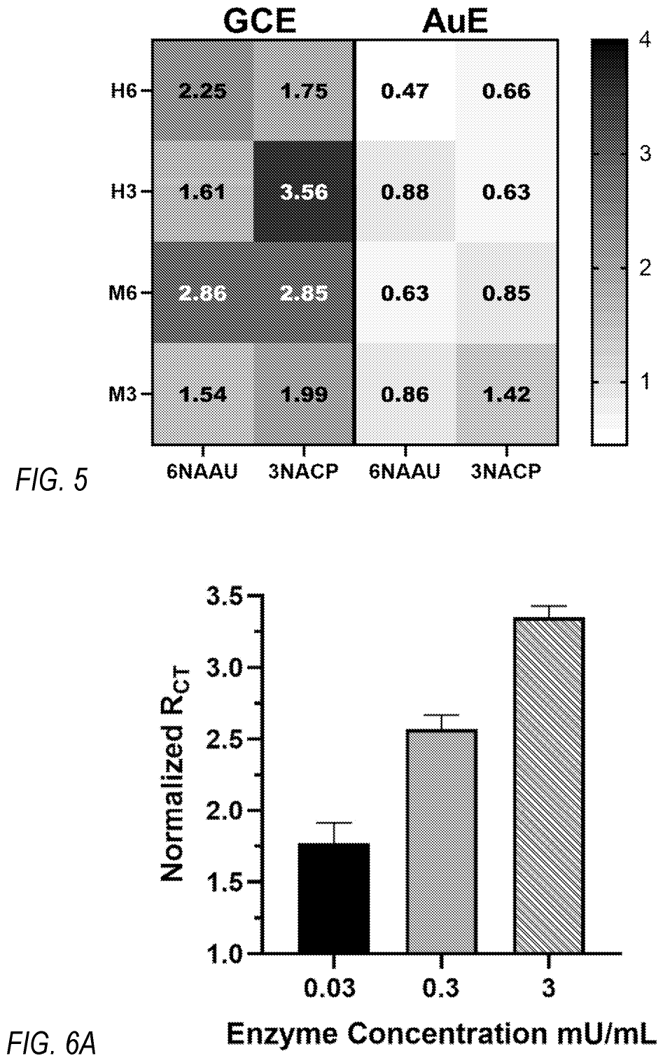

To further explicate the enzyme binding and reaction preferences on sialosides that derive from different animal, GCE-M6, GCE-M3, AuE-M6, and AuE-M3 were exposed to 6NAAU and to 3NACP and impedimetric analyses were performed. While GCE-M3 showed slight preference to 3NACP, GCE-M6 did not show preferential response to any of the NA (FIG. 5). This suggests that the binding preferential response is not only related to the position of the SA but also to its type or origin. Impedimetric control experiment was carried out by exposing 3NACP to GCE modified by same procedure with propyl amine. The exposure resulted in absence of significant change in R_{CT}, hence, indicating that the glycan is required for the recognition event. The enzymatic response using AuE-M6 showed a clear preference to 6NAAU as opposed to GCE-M6 (FIG. 5). This can be related to the interfacial interactions combined with interaction with the substrate that cause differentiation between the enzymes only on AuE. AuE-M3 showed low response to the unfavored enzyme 6NAAU that is in line with enzymatic preference. However, AuE-M3 showed an increase impedance when exposed to 3NACP, which is opposite signal trend to other AuE-based systems. This may be attributed to specific adsorption of 3NACP on AuE-M3 and not to enzymatic reaction. XPS analyses showed that there is a slight increase in the amide signals on AuE-M3 from adsorption of enzyme. This explains the observed increase in impedimetric response in the case of AuE-M3 response to 3NACP.

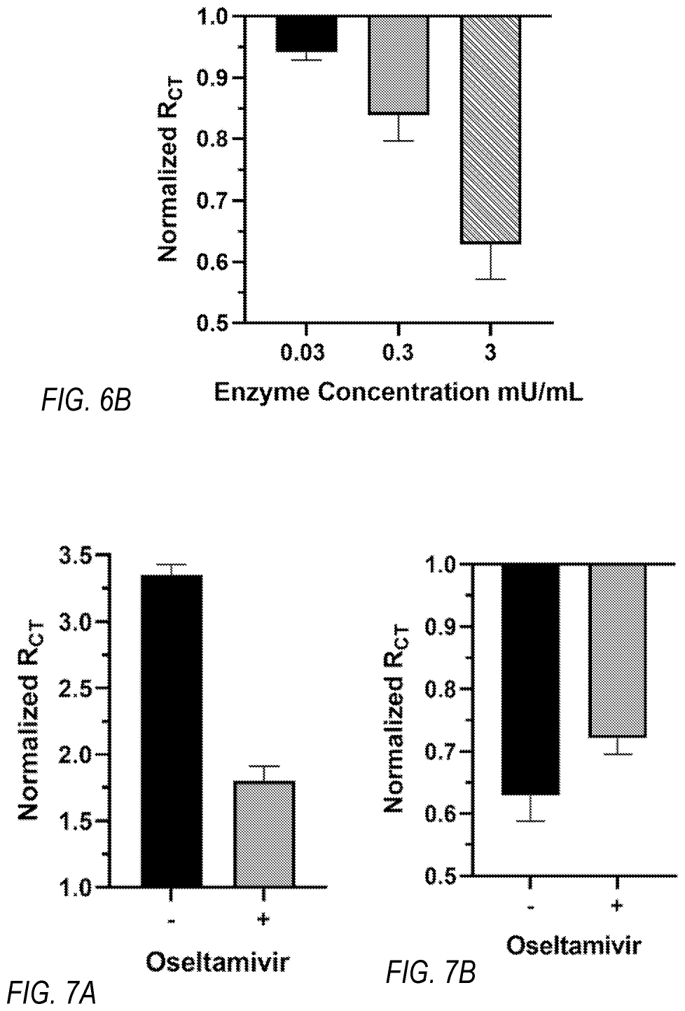

To evaluate the sensing sensitivity of the platform, GCE-H3 and AuE-H3 were exposed to 3, 0.3, and 0.03 mU/mL of 3NACP. The normalized response with different concentration of the NA was plotted (FIG. 6). These results show that there is a concentration-dependent behavior for the adsorption on GCE and the enzymatic reaction on AuE. The enzymatic reaction dependent system was insensitive to low concentrations of enzyme as opposed to GCE, hence, GCE based system has higher sensitivity for enzyme detection.

To evaluate the effect of NA inhibitor on the affinity to the modified GCE, GCE-H3 was used. GCE-H3 was exposed to 0.3 mU/mL 3NACP in the presence of 0, 0.1, and 1 μM of the antiviral drug Oseltamivir (FIG. 7A), which is a known NA inhibitor. Addition of 0.1 μM of Oseltamivir resulted in lower adsorption response of GCE-H3 to 3NACP. Increase in Oseltamivir concentration to 1 μM resulted in further decrease in response to the enzyme. The adsorption inhibition following exposure to Oseltamivir suggests that the competition on the catalytic site cause the decreased affinity to the sensory layer with the glycan. Combining these results with the deferential affinity to the proper substrate suggests that the enzyme major interaction is with substrate. There is a probability for additional interaction with the GCE that prevents the enzyme detachment from the interface as was observed by surface characterizations. Additionally, Evaluation of inhibition was performed on AuE-H3 with 3 mU/mL of 3NACP in presence and absence of 1 μM oseltamivir (FIG. 7B). The concentrations that were used on AuE-H3 were higher because there is lower sensitivity in the system when low concentrations are used. The decrease in response using the system of AuE-H3 in the presence of the inhibitor suggests that the enzyme inhibition activity can be sensed and studied utilizing the presented platforms.

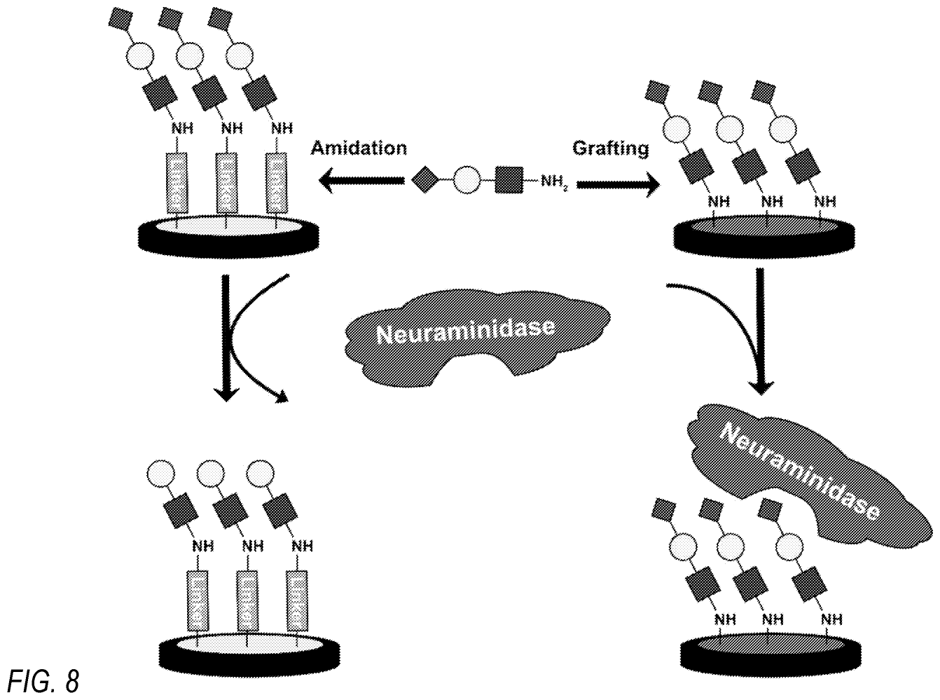

In this study, two platforms based on type of surface were developed for modification with four sialosides. The two platforms bearing the same substrate showed opposite impedimetric response when exposed to the same enzyme. Surface characterization showed that the type of behavior after exposure depends on the modified surface. This is in line with previous work that addressed the effect of surface characteristics on the phenomena occurring in the interfaces. In this work the modified AuE was sensitive to the enzymatic process while the modified GCE was selectively responsive to the presence of the enzyme binding on the surface (FIG. 8). The intensity of the response was correlated with the sialoside preference by enzyme regardless to the platform used. It was observed that the modified GCE has higher sensitivity compared to the modified AuE counterpart. Inhibition analyses showed that both platforms can detect inhibition in binding and reaction, however, the higher sensitivity of binding analyses suggest that GCE is the preferable method for design of NA inhibitors. Both systems are viable for sensing where AuE can be used for detection of the desialylation enzymatic reaction while GCE can be used for a more sensitive detection of NA presence.

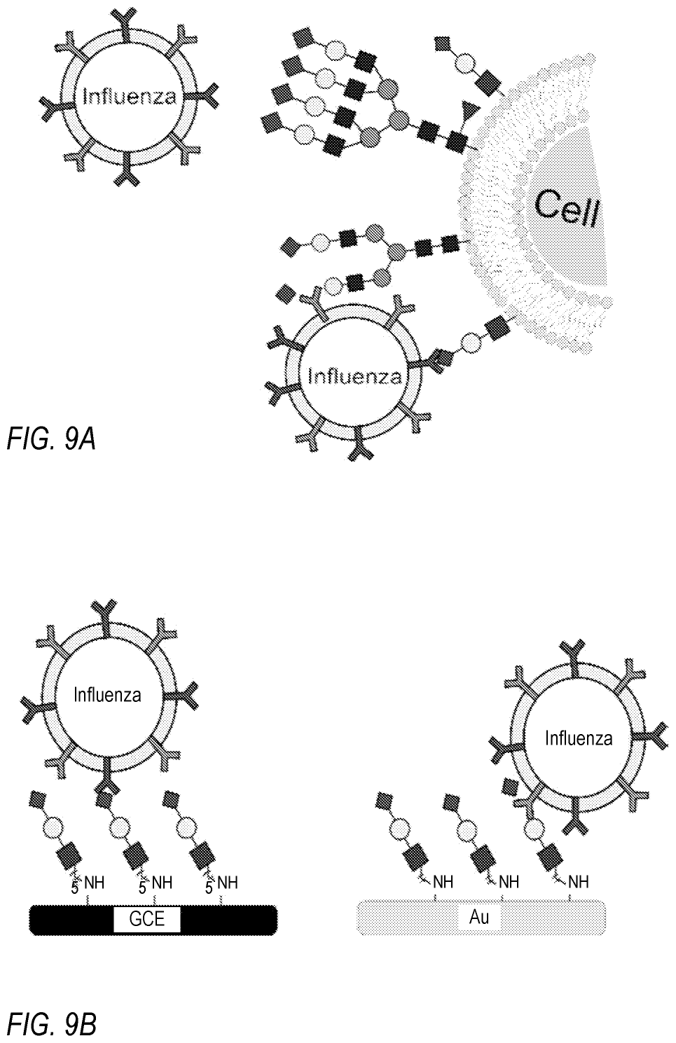

The system of the invention was utilized for impedimetric detection of various types of influenza NA based on the phenomena of binding and catalytic activity (FIG. 9). The results show electrochemically that the design system is sensitive to H1N1 and H3N2 NA, and that it can differentiate between the NA and distinguish them from bacterial NA by analyzing the response of the NA to 4 types of sialoside trisaccharide on two types of interfaces.

### Results and Discussion

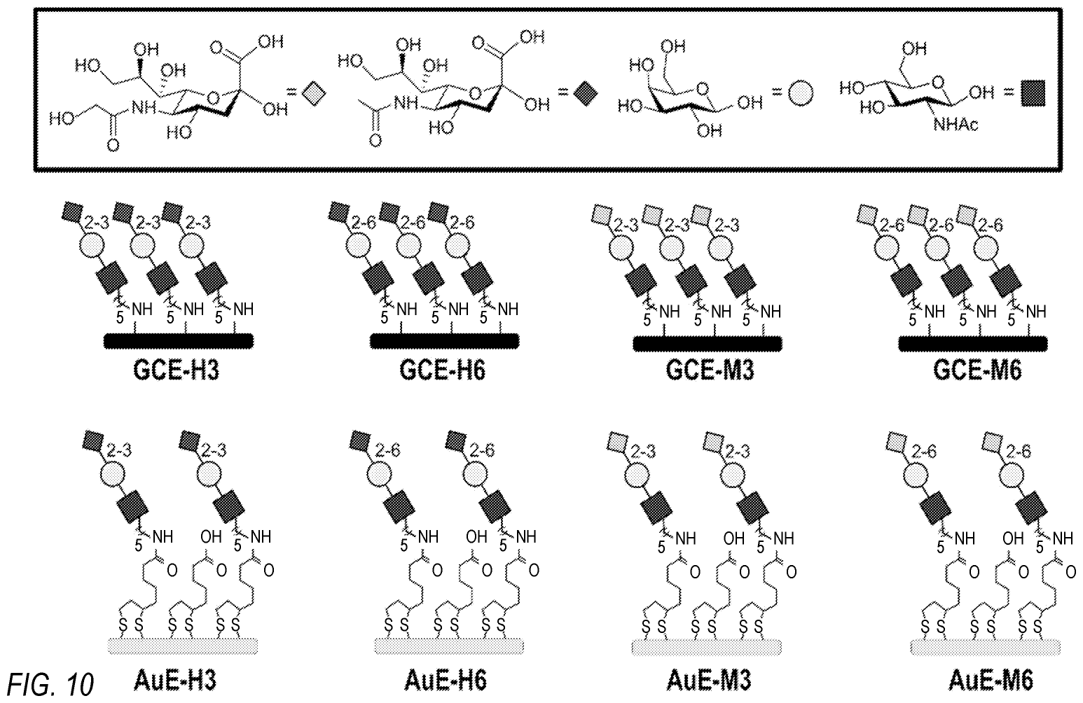

Two platforms modified with synthetic sialoside trisaccharides were developed for the detection of neuraminidase based on binding and enzymatic activity of bacterial NA. The first interface is produced by electro-grafting glassy carbon electrodes (GCE) with Trisaccharide with amine linker at the reducing end to give GCE-H3, GCE-H6, GCE-M3, and GCE-M6 (FIG. 10). The second interface is produced by coupling the amine terminated trisaccharides to Lipoic acid (LPA) monolayer on Au electrode (AuE) to give AuE-H3, AuE-H6, AuE-M3, and AuE-M6 (FIG. 10). The eight modified interfaces showed ability to differentiate impedimetrically between bacterial NA by the variation of response produced in each electrode type, therefore, the have potential in differentiation between viral NA based on affinity and catalytic activity.

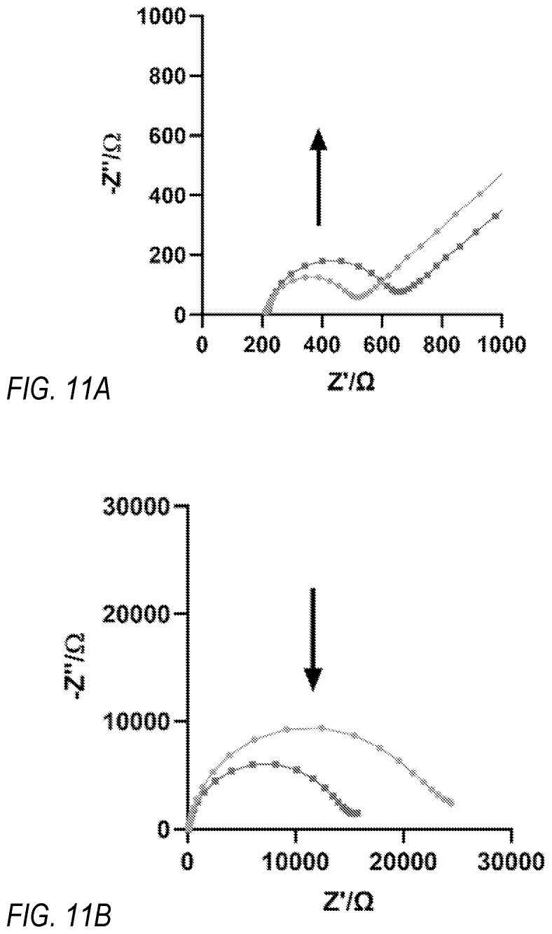

To determine if the systems respond impedimetrically to viral NA in similar manner as to bacterial NA, EIS measurement was performed using GCE-H3 before and after exposure to H1N1 NA. The exposure to the enzyme resulted in increase of charge transfer resistance (RCT) (FIG. 11A), which is in line with our previous observation for NA binding. EIS measurements were also performed using AuE-H3 before and after exposure to H1N1 NA. This exposure resulted in a decrease of RCT, which is in line with our previous observation of NA enzymatic activity on AuE modified with sialosides. To confirm the observation of viral NA binding and activity, XPS analyses were performed on modified Au and GC.

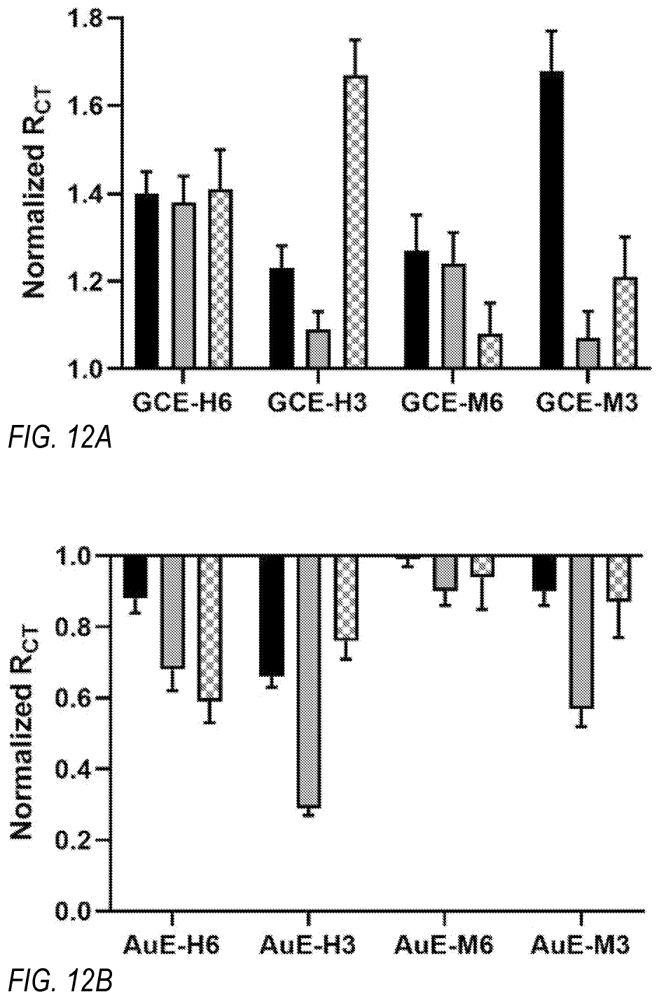

For comparative study of viral NA response by the various platforms, each system was exposed to H1N1 and H3N2 NA and the impedimetric response was recorded, Normalized, and plotted (FIG. 12). Modified GCE responded in increase of RCT in except in the case of GCE-H3 and GCE-M3 with H3N2 NA (FIG. 12A). Unlike the case of H3N2, GCE-H3 and GCE-M3 responded to H1N1 NA. This indicates that these two platforms can detect H1N1 NA but cannot detect H2N3. GCE-H6 and GCE-M6 responded to both H1N1 and H3N2 with similar intensity, indicating that the two platforms can detect both viral NA but cannot differentiate between them. Combination of the responses can be used to both detect the viruses and differentiate between them. Modified AuE were exposed to the two enzymes and the normalized RCT was recorded (FIG. 12B). AuE-M6 showed no response to H1N1 while responded to H3N2. This indicates the AuE-M6 can be used to detect H3N2 but not H1N1. AuE-H3, AuE-H6, and AuE-M3 responded to both H1N1 and H3N2. In all these cases the enzymatic activity sensing was higher for H3N2 indicating faster enzymatic activity in the presented interface. Both H3N2 and H1N1 NA showed the highest activity on AuE-H3, which is in line with previous works on influenza NA substrate preference. This indicates that the presented system can be used also to study viral NA enzymatic activity and not only differentiate between the NA for sensing applications.

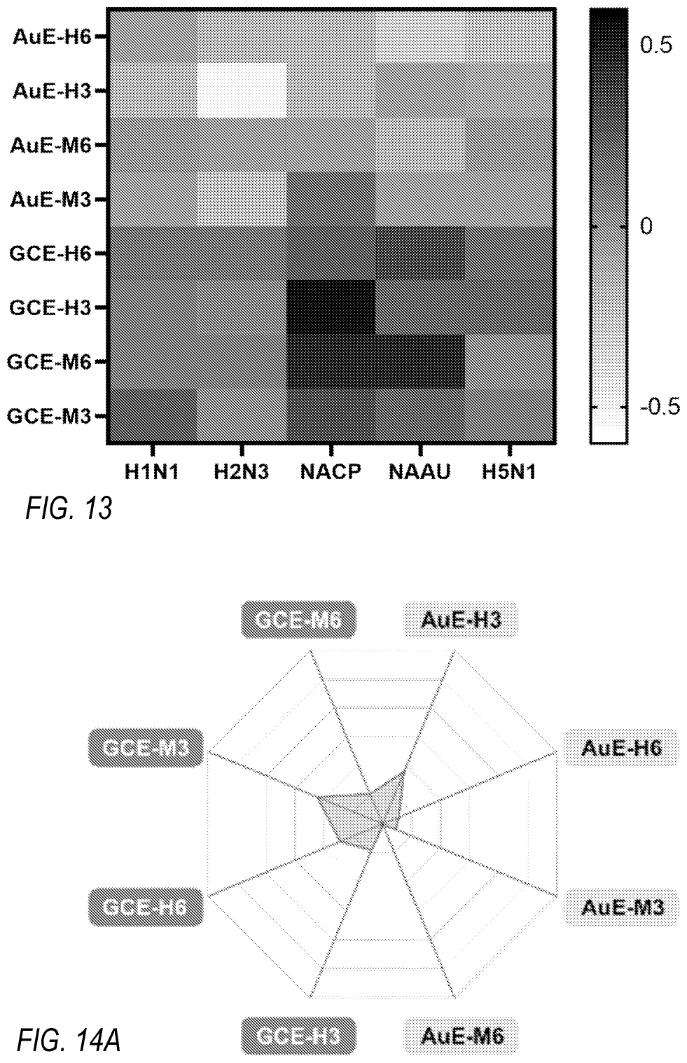

To compare between the responses, Log (normalized RCT) was taken for both influenza NA response in this study and bacterial NA, which are Clostridium perfringens NA (3NACP) and Arthrobacter ureafaciens NA (6NAAU). The calculated results were plotted on a heatmap (FIG. 13). The results show that the bacterial NA has higher response for binding. This can be as result from the difference in NA exposed surface that cause stronger binding by bacterial NA. In the case of activity on AuE the viral NA show higher preferential activity for the correct substrate than bacterial NA. when looking at the structure of viral Neuramindases (PDBid 7S0I and 4H52 for H1N1 and H3N2 respectively), the pocket is located close to the surface of the enzyme with low hydrophobicity surrounding the pocket. This is different from bacterial NA where the pocket is located deeper in the enzyme and the surrounding interface is hydrophobic. The structural differences between the enzyme dictates its affinity to the interface and ability to perform reaction on solid-liquid interface. It is important to note that combination of the eight interfaces for detection of NA showed ability to differentiate between the 4 NA proving to be a powerful tool in infection type detection based on NA activity.

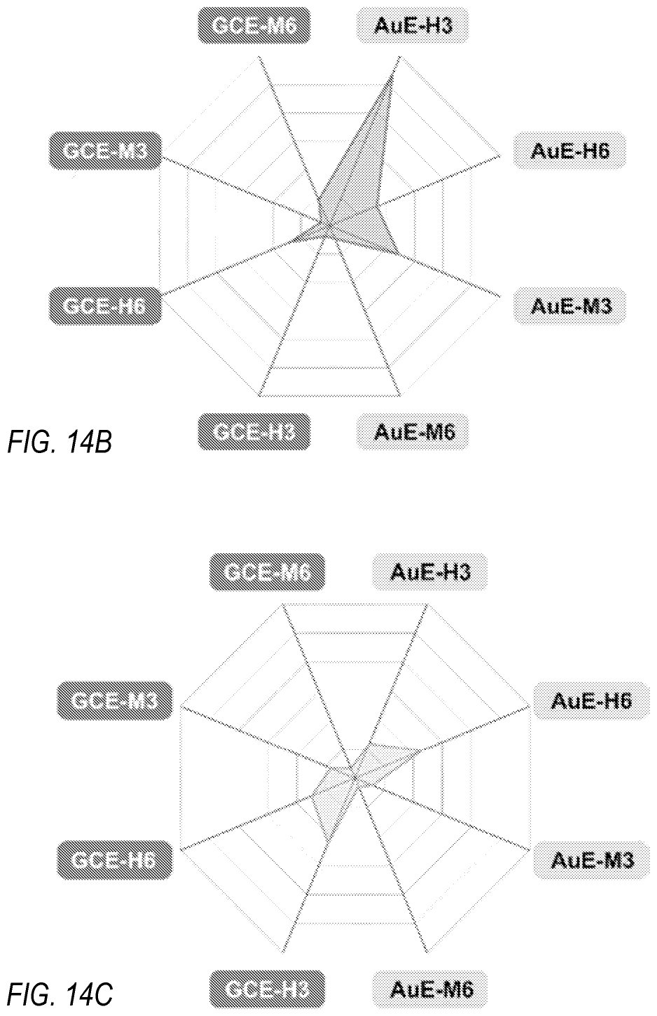

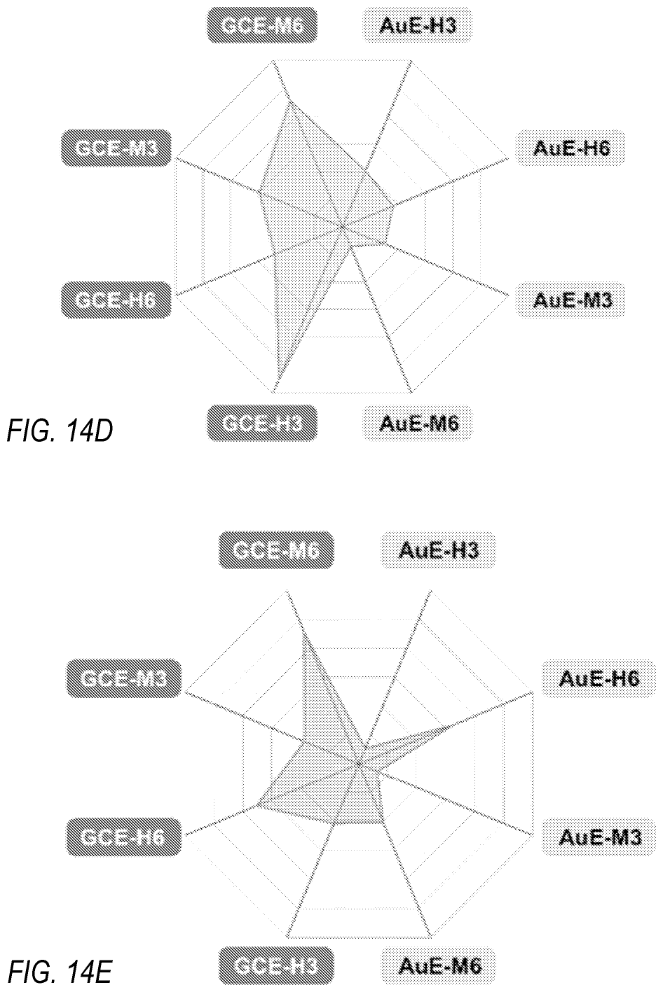

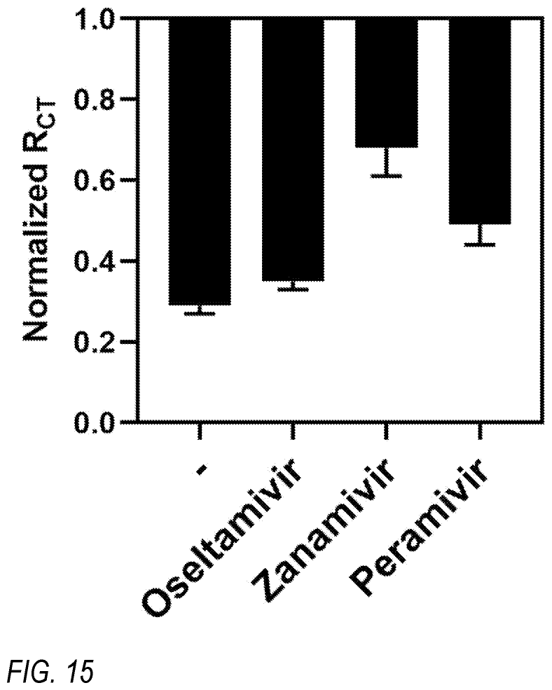

A Radar plot of |Log (Normalized RCT)| for the response of H1N1, H3N2, H5N1, NACP, and NAAU with the presented sensory layers is demonstrated in FIGS. 14A-E. FIG. 15 provides the normalized RCT response of AuE-H3 to H3N2 NA in presence and absence of Viral NA inhibitors.

### CONCLUSION

In this work, the inventors have demonstrated the ability to detect and differentiate between viral neuraminidase based on preferential response with sialoside decorated interfaces, which relies on affinity differences to the substrate on an electrode. Results show that binding and catalytic activity of the enzyme was detected by location of the catalytic pocket and surrounding enzyme interface. This causes viral NA, where the pocket is at the enzyme interface, to perform reaction faster on the surface while the bacterial NA, where the pocket is deeper with hydrophobic surrounding interface, to bind stronger the sialosides.

As demonstrated, glassy carbon and Au electrodes were modified by four types of sialylated trisaccharides, which differ by the type of SA and is galactose bound regiochemistry. The modifications were verified by EIS, CPD, and CA analyses, which resulted in changes of R_{CT}, surface potential, and hydrophobicity. The systems were exposed to two NAs with different regiochemistry preferences. This resulted in a preferential signal on the various substrates. Surface characterizations, such as XPS and CPD, support that the changes in impedimetric signals were a result of enzyme adsorption on the surface in the case of GCE and enzymatic reaction in the case of Au. Increase of EIS signal related to the adsorption of the enzyme and decrease for enzymatic reaction proved to be concentration dependent and substrate specific based on glycosidic linkage and type of sialic acid. Additionally, enzyme adsorption and enzymatic reaction in the interface were reduced by the presence of the NA inhibitor Oseltamivir presence. The collective results suggested that the designed platform can be used for rapid detection of NA by an array of small glycan and evaluation of NA inhibitors using both enzymatic sedimentation and enzymatic reaction on the modified interfaces.

1.-46. (canceled)

47. An electrode material in a form of a sialoside, wherein the sialoside comprises a sialic acid associated to a glycan selected from monosaccharides, disaccharides, tri saccharide and oligosaccharides.

48. The electrode material according to claim 47, wherein the glycan comprises or consists β-D-Gal-(1-4)-β-D-GlcNAc-(1-4))-1-X, wherein Gal is galactose, Glc is glucose and X is a group selected from C₂-C₁₀alkyl, C₂-C₁₀alkenyl, C₂-C₁₀alkynyl, C₆-C₁₀arylene, and C₂-C₁₀alkyl-C₆-C₁₀aryl, each independently being substituted by an atom or a group of atoms selected from halogen (I, Br, Cl or F), hydroxy (OH), thiol (SH), disulfide (—S—S—), cyano (CN), nitro (NO₂), azide (N₃), amine (NH₂), and carboxy (COO).

49. The electrode material according to claim 47, wherein the glycan comprises or consists a sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, and β-D-Gal-(1-4)-β-D-GlcNAc-(1-4))-1-octylamine.

50. The electrode material according to claim 47, wherein the sialoside is a trisaccharide comprising a sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, and β-D-Gal-(1-4)-β-D-GlcNAc-(1-4))-1-octylamine, and a moiety derived from N-acetylneuraminic acid (Neu5Ac) or N-glycolylneuraminic acid (Neu5Gc).

51. The electrode material according to claim 47, being a sialoside of a structure selected from:

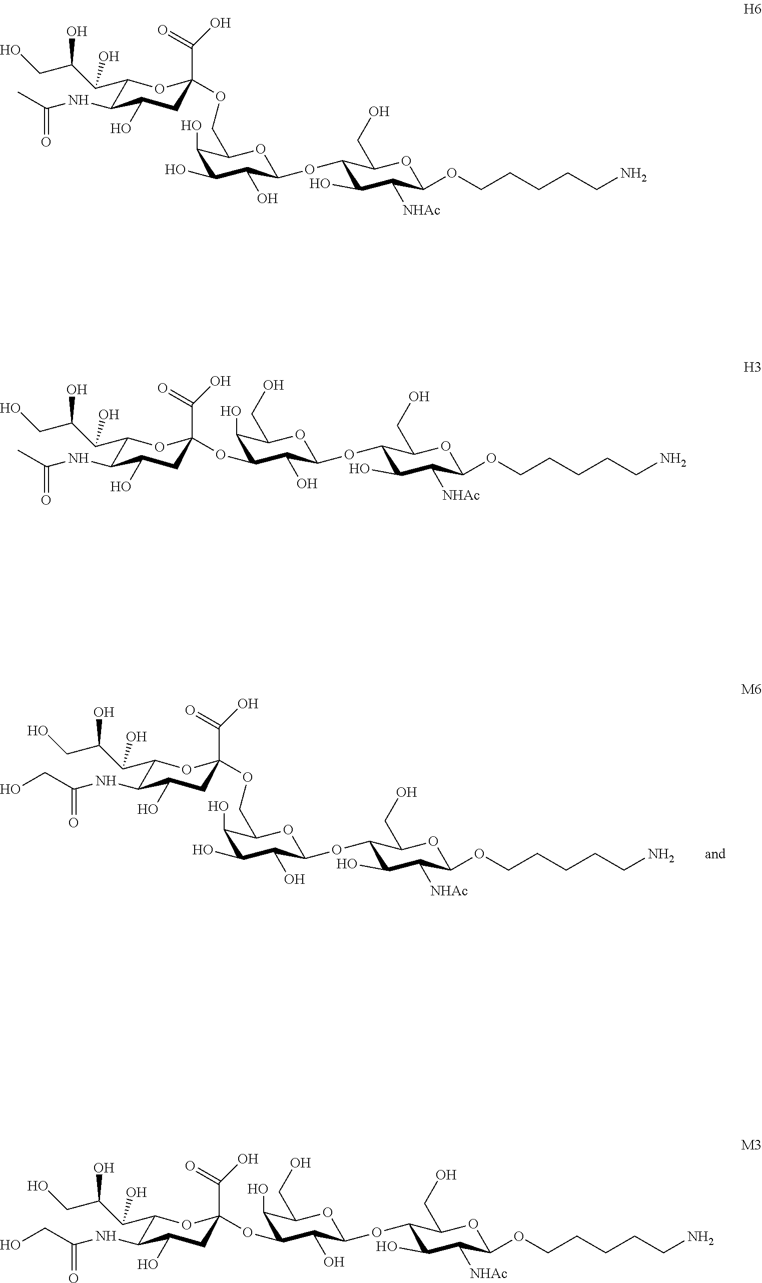

52. An electrode having a surface modified with an electrode material according to claim 47.

53. The electrode according to claim 52, being a gold or a GCE electrode or an electrode for use in electrochemical impedance spectroscopy (EIS).

54. The electrode according to claim 52, wherein the sialoside population is a trisaccharide, the sialoside comprising a glycan comprising Gal and/or Glc.

55. The electrode according to claim 54, wherein the glycan comprises or consists β-D-Gal-(1-4)-β-D-GlcNAc-(1-4))-1-X, wherein Gal is galactose, Glc is glucose and X is a group selected from C₂-C₁₀alkyl, C₂-C₁₀alkenyl, C₂-C₁₀alkynyl, C₆-C₁₀arylene, and C₂-C₁₀alkyl-C₆-C₁₀aryl, being substituted by an atom or a group of atoms selected from halogen (I, Br, Cl or F), hydroxy (OH), thiol (SH), disulfide (—S—S—), cyano (CN), nitro (NO₂), azide (N₃), amine (NH₂) and carboxy (COO).

56. The electrode according to claim 55, wherein the glycan comprises or consists β-D-Gal-(1-4)-β-D-GlcNAc-(1-4))-1-X-amine, wherein X is selected from C₂-C₁₀alkyl, C₂-C₁₀alkenyl, C₂-C₁₀alkynyl, C₆-C₁₀arylene, and C₂-C₁₀alkyl-C₆-C₁₀aryl.

57. The electrode according to claim 56, wherein X is C₂-C₁₀alkyl.

58. The electrode according to claim 54, wherein the glycan comprises or consists a sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, and β-D-Gal-(1-4)-β-D-GlcNAc-(1-4))-1-octylamine.

59. The electrode according to claim 52, associated with a sialoside of a structure selected from:

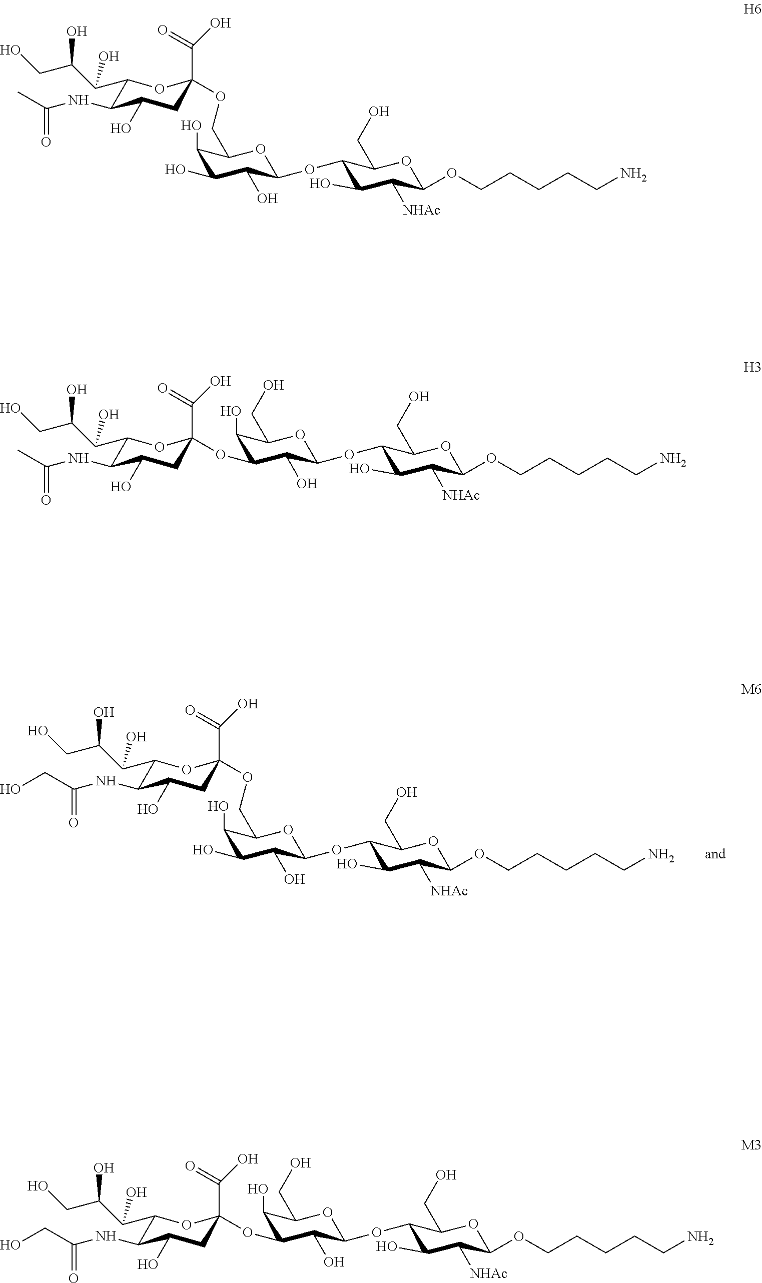

60. An electrode for use in an impedance measuring unit, the electrode comprising a biosensing surface associated with a sialoside selected from

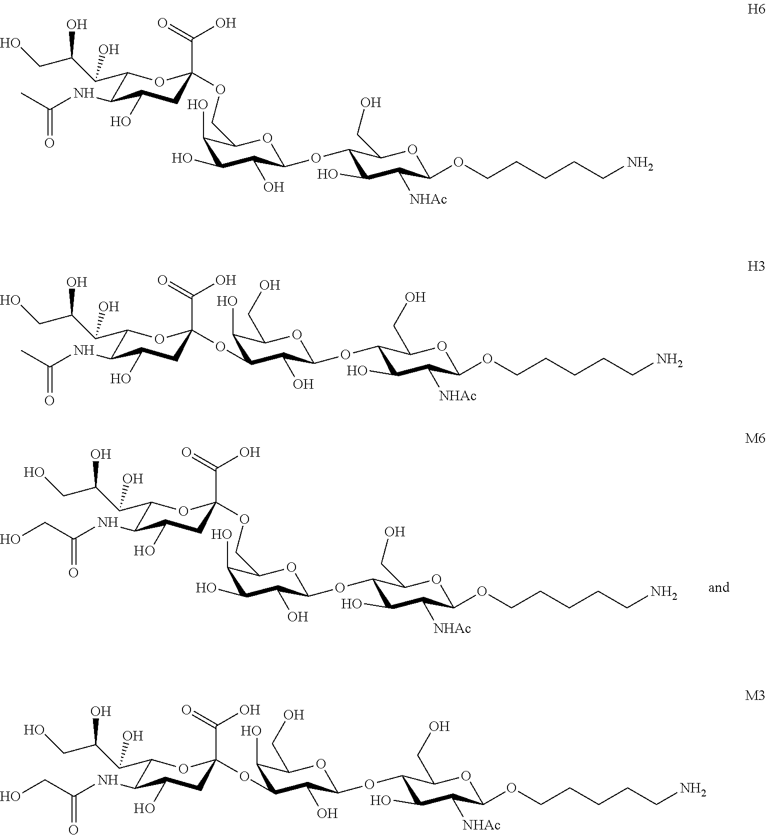

61. A detection device comprising an electrode having a surface modified with a sialoside comprising a sialic acid associated to a glycan selected from a monosaccharide, a disaccharide, a trisaccharide and an oligosaccharide.

62. A method for determining an interaction between a sugar implemented as a sialylated glycan on an electrode surface and an agent capable of chemically interacting with the sialylated glycan, the method comprising contacting the electrode surface modified with the sialylated glycan with a sample containing or suspected of containing said agent and determining a change in an impedance signal generated from a base signal obtained for a control sample, wherein a change in the signal indicates a chemical interaction between said sialylated glycan and said agent.

63. The method according to claim 62, for screening for enzyme inhibitors.

64. The method according to claim 63, wherein the enzyme inhibitor is an antiviral, antibiotic, anticancer drug.

65. The method according to claim 63, wherein the enzyme inhibitor is an inhibitor of neuraminidases (NA).

66. The method according to claim 63, wherein the enzyme inhibitor is an inhibitor of sialyltransferase.
